(19) United States
(12) Patent Application Publication
(10) Pub. No.: US 2005/0084909 A1
(43) Pub. Date: Apr. 21, 2005

(54) Antigen measuring device and method thereof, an antibody chip package and a pallet

(76) Inventors: Kenichi Uchiyama, Kanagawa-ken (JP); Kayoko Oomiya, Kanagawa-ken (JP); Isao Kishimoto, Kanagawa-ken (JP); Masami Hirata, Kanagawa-ken (JP); Hideo Eto, Kanagawa-ken (JP); Ichiro Tono, Kanagawa-ken (JP); Ikuo Uematsu, Kanagawa-ken (JP)

Correspondence Address: Finnegan, Henderson, Farabow,;Garrett & Dunner, L.L.P., 1300 I Street, N.W., Washington, DC, 20005-3315, US

(21) Appl. No.: 10/928,319

(22) Filed: Aug. 30, 2004

(30) Foreign Application Priority Data
Aug. 29, 2003 (JP) P2003-307868

(51) Int. Cl. C12Q 1/68; G01N 33/53; C12M 1/34

(52) U.S. Cl. 435/7.1; 435/287.2

(57) ABSTRACT

An antigen measuring device has an antibody chip. The antibody chip has a substrate. A pair of gratings are formed on the substrate. An antibody-fixed layer surrounded by a wall is formed between the gratings. A light emitting element emits a light beam toward the first grating. A light receiving element receives the light beam which propagates through the substrate under the antibody-fixed layer and is outputfrom the second grating.

### CROSS REFERENCE TO RELATED APPLICATION

This application is based upon and claims the benefit of priority from prior Japanese Patent Application No. 2003-307868 filed on Aug. 29, 2003, the entire contents of which are incorporated herein by reference.

### BACKGROUND

1. Technical Field

The present invention relates to an antigen measuring device, in particular, an antigen measuring device for measuring a small amount of antigen such as a protein contained in a liquid solution. The present invention further relates to a method for measuring antigen, a pallet, and an antibody chip package.

2. Description of the Related Art

The Enzyme-Linked Immuno Sorbent Assay (ELISA) method is known as a method to measure minor constituents. The ELISA method utilizes a specific reaction between an antibody and an antigen. An immune sensor with a light guide using the ELISA method is shown in Japanese Patent Publication (Kokai) No.08-285851. This immune sensor has a pair of gratings on a surface of a substrate. One is for inputting a light beam into the substrate. The other is for outputting the light beam from the substrate. A light guide layer is formed between these two gratings. An antibody-fixed film is further formed on the light guide layer. When a liquid solution containing antigens to be measured is contacted with the antibody-fixed layer, the antigen is bound to an antibody of the antibody-fixed layer. When a fluorescent labeled antibody is further added, an immune complex of the antibody, the antigen, and the fluorescent labeled antibody is formed on the surface of the substrate. Under the existence of the immune complex, a laser beam is inputted into the light guide via the grating. The inputted laser beam generates evanescent waves while propagating the light guide. The evanescent waves excite the immune complex so that the immune complex emits fluorescent light. Therefore, the amount of biological molecules in the liquid solution can be measured based on the intensity of the fluorescent light received by a light receiving element.

However, the sensitivity of the variation is limited under the conventional immune sensor, which has a difficulty in measuring a small amount of biological molecules.

### SUMMARY

One aspect of the present invention is an antigen measuring device. The antigen measuring device comprises an antibody chip which includes a substrate, a first grating on the surface of the substrate, a second grating on the surface of the substrate, an antibody-fixed layer on the surface of the substrate between the first and the second gratings, and a wall on the surface of the substrate, wherein the wall surrounds the antibody-fixed layer. The antigen measuring device further comprises a light emitting element to emit a light beam toward the first grating and a light receiving element to receive the light beam after it propagates through the substrate under the antibody-fixed layer and is output from the second grating.

In another aspect consistent with the present invention, there is provided a method of measuring antigen. The method comprises dropping a first liquid solution containing an antigen on an antibody-fixed layer formed on a substrate of an antibody chip to form a complex of an antibody and the antigen, which antibody chip includes first and second gratings on the substrate on either side of the antibody-fixed layer; dropping a second liquid solution containing a secondary antibody labeled by a labeling enzyme to form a complex of the antibody, the antigen, and the secondary antibody; firstly measuring the intensity of a first light beam which propagates toward the first grating, through the substrate, and from the second grating; dropping a coloring reagent solution on the antibody-fixed layer to produce an enzyme reaction product colored by a reaction with the labeling enzyme; secondarily measuring the intensity of a second light beam which propagates toward the first grating, through the substrate, and from the second grating; and measuring the antigen based on the intensities of the received light beams.

Another aspect of the present invention is a pallet with plural indentations to respectively set a plurality of antibody chips, wherein the distance between the center of the indentations in the pallet is 9 mm.

Another aspect of the present invention is an antibody chip package. The antibody chip package comprises a chip container to set plural antibody chips at a constant interval, a desiccant to keep the antibody chips dry, and a package cover bound to the chip container.

Additional features and advantages consistent with the invention will be set forth in part in the description which follows, and in part will be obvious from the description or claims, or may be learned by practice of the invention.

It is to be understood that both the foregoing general description and the following detailed description are exemplary and explanatory only and are not restrictive of the invention as claimed.

The accompanying drawings, which are incorporated in and constitute a part of this specification, illustrate several aspects of the invention and together with the description, serve to explain the principles of the invention.

### BRIEF DESCRIPTION OF THE DRAWINGS

FIG. 1 is a schematic diagram of a protein measuring device.

FIG. 2 is a plan view of four pallets connected to each other.

FIG. 3 is a plan view of an antibody chip package.

FIG. 4 is a cross section of the antibody chip package shown in FIG. 3.

FIG. 5 is a plan view of an antibody chip.

FIG. 6 is a cross section of the antibody chip shown in FIG. 5.

FIG. 7 is a flow chart showing a method of manufacturing an antibodychip.

FIGS. 8A-8C, 9A and 9B show a method for manufacturing an antibody chip.

FIG. 10 is a flow chart showing a method of measuring a density of protein.

FIGS. 11A-11D show immune and chemical reactions in a method for measuring an amount of protein.

### DETAILED DESCRIPTION

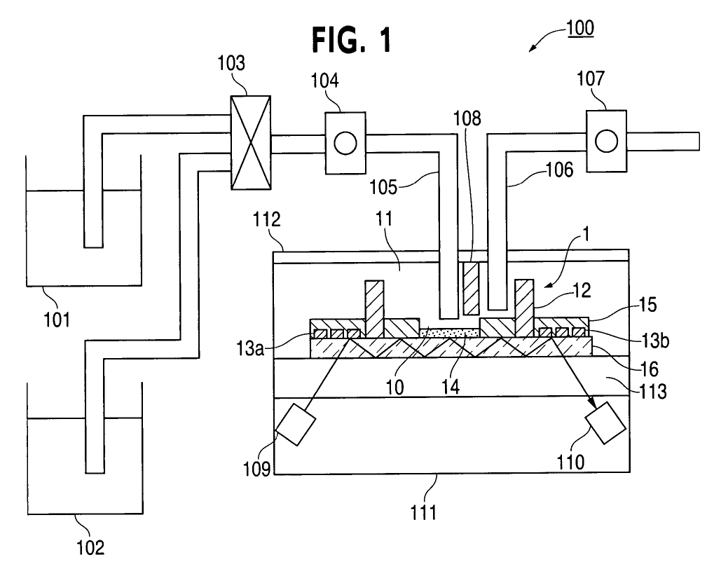

Embodiments consistent with the invention are explained next with reference to FIGS. 1 to 12. Wherever possible, the same reference numbers are used throughout the drawings to refer to the same or similar elements. Scale or proportions of elements shown in these figures are for purposes of illustrationand may discord from reality.

Protein Measuring Device

As one example of an antigen measuring device in consistent with the present invention, this embodiment shows a device for measuring protein. FIG. 1 shows a protein measuring device 100 having an antibody chip 1. Antibody chip 1 is provided with a substrate 16, an incident grating (first grating) 13a, an outputting grating (second grating) 13b, an antibody-fixed layer 14 between gratings 13a and 13b, and a cell wall 12 surrounding antibody-fixed layer 14. The surrounded space is called cell 11 in this embodiment.

Protein measuring device 100 further includes a light emitting element 109 which emits a light beam toward incident grating 13a and a light receiving element 110 to receive a light beam. After being emitted, the light beam propagates through substrate 16 and is output from outputting grating 13b. The surface of substrate 16, including gratings 13a and 13b except a reaction hole 10 where antibody-fixed layer 14 is formed, is covered with a fluoroplastic film 15.

Protein measuring device 100 further comprises an injection tube 105 to inject a liquid solution into cell 11 and an evacuation tube 106 to remove the injected liquid solution from cell 11. Injection and evacuation tubes 105 and 106 are respectively connected to an injection pump 104 and an evacuation pump 107. The outlet of injection tube 105 is preferably positioned just above antibody-fixed layer 14 to surely drop a liquid solution on antibody-fixed layer 14. One end of evacuation tube 106 is preferably positioned beside cell wall 12.

Since fluoroplastic film 15 is water-repellent, liquid solution overswelling reaction hole 10 flows toward cell wall 12 so that the overswelled liquid solution is evacuated through evacuation tube 106. A container lid 112 covers antibody chip 1. Since injection tube 105 and evacuation tube 106 are fixed with lid 112, the positions of injection tube 105 and evacuation tube 106 are stabilized. A syringe pump or the like may be used as injection pump 104. A bimorph pump or the like may be used for evacuation pump 107.

Protein measuring device 100 also comprises a first solution container 101, a second solution container 102, and a valve 103 connected to injection pump 104. Solution to be injected can be selected with valve 103.

In this embodiment, first solution container 101 contains washing liquid including a surface active agent, and second solution container 102 contains a buffering solution. Valve 103 can automatically select between the solutions according to measurement procedures. Injection pump 104 may be configured to operate only when antibody chip 1 is installed in protein measuring device 100 to avoid injection of solutions without antibody chip 1.

Since a light beam from light emitting element 109 is usually a laser beam or the like, it could be harmful to irradiate an eyeball by the light beam. To avoid such irradiation, light emitting element 109 may be configured to operate only when antibody chip 1 is closed by lid 112.

Additionally, a light blocking plate 108 may project from lid 112 toward antibody chip 1 to block a light beam traveling outside of substrate 16 so that light receiving element 10 mainly receives the light beam emitted from light emitting element 109 and propagating inside substrate 16.

Pallet

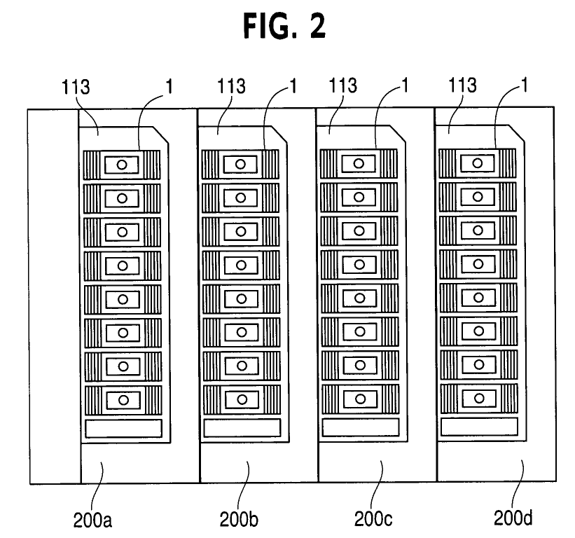

FIG. 2 shows a plurality of pallets 113, each pallet 113 having plural indentations to place antibody chips 1 therein for use in a protein measuring device as shown, e.g., in FIG. 1.

As shown in FIG. 2, each unit 200a-d comprises one pallet 113 and antibody chips 1 set therein. In this embodiment, each pallet 113 has eight indentations to respectively set antibody chips 1. As an example, the distance between the center of the indentations may be arranged to be 9 mm. As shown, the units 200a-d can be connected to each other200a-dwhen they are installed in protein measuring device 100. While FIG. 2 illustrates four units 200a-d, different numbers of units may be used, e.g. three units or five units.

When a unit is connected to other units, it is preferred to distinguish the units from each other. For that purpose, each pallet 113 may have identification (not shown). Protein measuring device 100 can recognize the identification and automatically select a measurement procedure according to that identification.

Antibody Chip Package

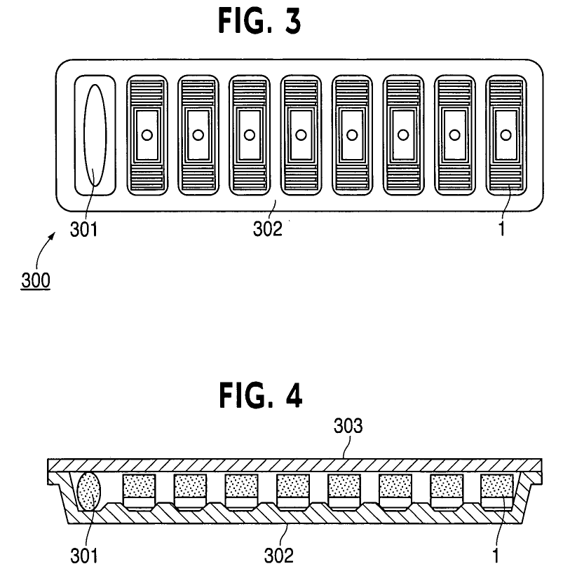

An antibody chip package 300 is shown in FIGS. 3 and 4. Antibody chip package 300 comprises a chip container 302 to set plural antibody chips 1 so that the distance of the center of antibody chips 1 remains constant. Antibody chip package 300 may also comprise a desiccant 301 to keep antibody chips 1 dry and a package cover 303 bound to chip container 302by, e.g., thermo-compression.

Chip container 302 may be made from polystyrene or the like. Desiccant 301 may be silica gel or the like. Package cover 303 may be made from polyethylene terephthalate (PET), polystyrene, a laminated film, or the like.

Multi pipettes or operation tools for microwell plates generally have eight pipettes arranged at 9 mm intervals. In order to improve compatibility with such instruments, antibody chip package 300 is preferably capable of containing eight antibody chips 1 arranged at 9 mm intervals.

Likewise with respect to the embodiment shown in FIG. 2, pallet 113 may be compatible with such instruments, so that installment and arrangement of antibody chips 1 from antibody chip package 300 to pallet 113 is easier. When removing used antibody chips 1 from pallet 113, an operator can easily discard them without contaminating the operator's hands by just returning the used antibody chips 1 to antibody chip package 300.

Antibody Chip

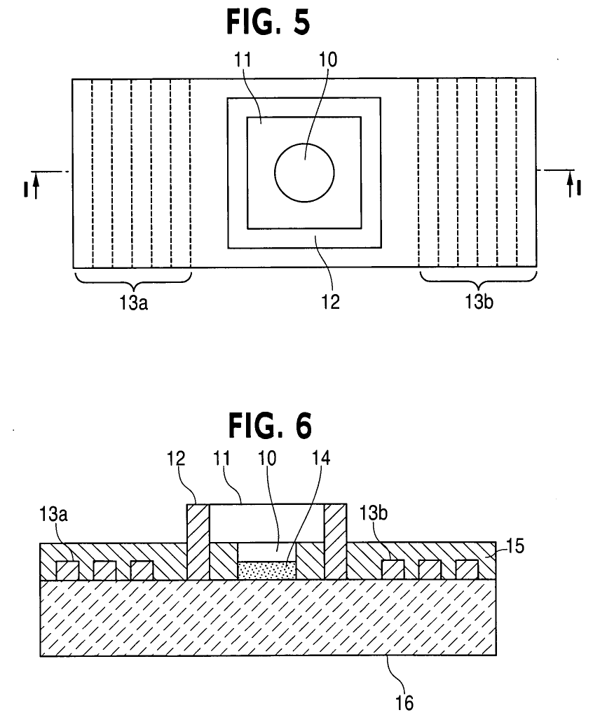

FIGS. 5 and 6 show an antibody chip 1. The antibody chip comprises substrate 16, which may be made of glass, incident grating (first grating) 13a and outputting grating (second grating) 13b formed on each end of the surface of substrate 16, and an antibody-fixed layer 14 between the gratings.

Antibody chip 1 further comprises cell wall 12 surrounding antibody-fixed layer 14. The surrounded space is called cell 11. The surface of substrate 16 including gratings 13a and 13b, and the bottom face of cell 11 except reaction hole 10 is covered with fluoroplastic film 15 as mentioned above.

Cell wall 12 may preferably be made from colored acrylic such as black acrylic. Fluoroplastic film 15 may preferably be made from fluorocarbon resin with light blocking effect to prevent an inputted light beam from leaking to the outside of antibody chip 1. Colored acrylic such as black acrylic may be used for fluoroplastic film 15. Incident and outputting gratings 13a and 13b may be made from Titanium Oxide (TiO₂).

Antibody-fixed layer 14 may have a structure with an antibody fixed by a crosslinked polymer. A polymer having hydrogen-bondable functional groups such as photocrosslinkable polyvinyl alcohol may be used as the cross linked polymer.

Method For Manufacturing An Antibody Chip

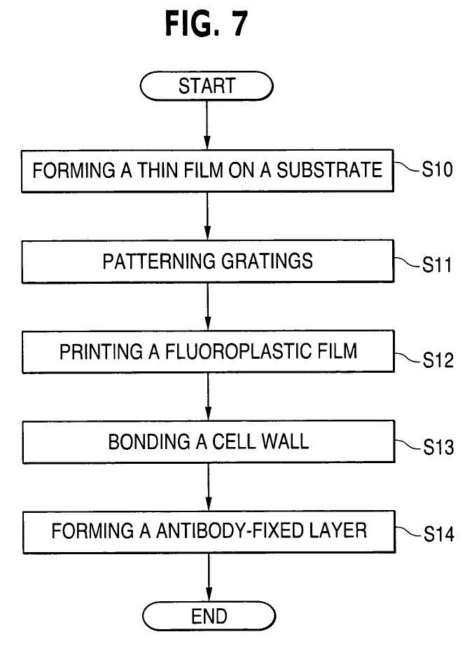

One embodiment of a method for manufacturing an antibody chip 1 according to aspects of the invention is explained next with reference to FIGS. 7 to 9.

FIG. 7 is a flowchart showing steps of manufacturing an antibody chip 1 according to aspects of the present invention. In a first step (S10), a thin film is formed on a substrate. In subsequent steps, gratings are patterned (S11), a fluoroplastic film is printed (S12), a cell wall is bonded to the substrate (S13), and an antibody-fixed layer is formed (S14). As one of ordinary skill in the art would appreciate, other steps may occur before, during, or after the steps shown in FIG. 7 or some of the steps shown in FIG. 7 may be re-ordered or deleted without departing from aspects of the present invention.

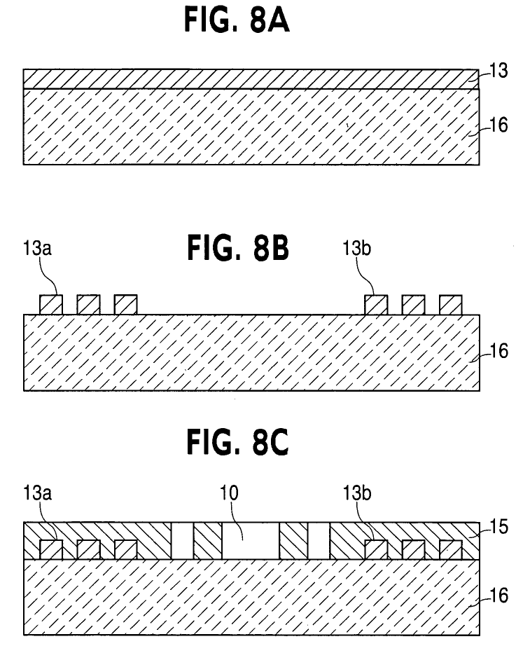

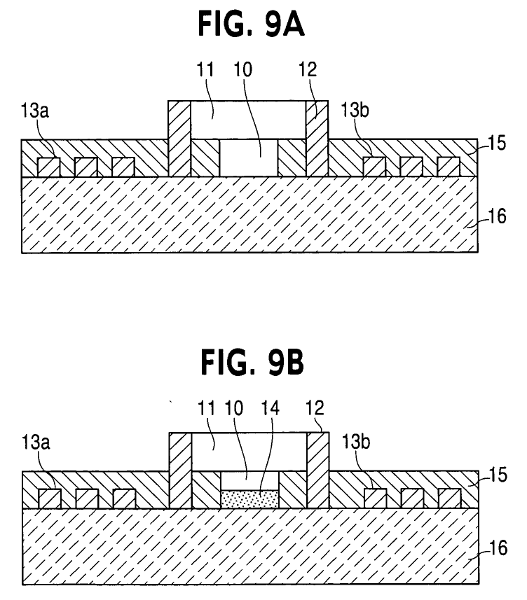

FIGS. 8A-8C and 9A-9B illustrate, respectively, steps S10-S14 according to the flowchart of FIG. 7.

(S10)

As shown in FIG. 8A, a thin film 13 is formed on a surface of substrate 16 (which may be made of borosilicic acid glass) by sputtering TiO₂.

(S11)

As shown in FIG. 8B, thin film 13 is partly etched by photo-etching to form incident grating (first grating) 13a and outputting grating (second grating) 13b on each end of substrate 16.

(S12)

As shown in FIG. 8C, a colored fluoroplastic film 15 with light blocking effect is formed by printing on the surface of substrate 16 including gratings 13a and 13b except reaction hole 10 and an area where cell wall 12 is to be bonded.

(S13)

As shown in FIG. 9A, cell wall 12, which may be made from black acrylic, is provided to surround reaction hole 10. Cell wall 12 may be bonded on substrate 16 with, e.g., an ultra violet cure adhesive (not shown), so that cell 11 is formed.

(S14)

As shown in FIG. 9B, antibody-fixed layer 14 is formed in reaction hole 10. Specifically, the surface of substrate 16 in reaction hole 10 is modified by an amino group. This modification may be accomplished, e.g., by using an aminosilane, which is a silane coupling agent. Next, antibodies are fixed to the surface of substrate 16 in reaction hole 10 by cross-linking using, e.g., glutaric aldehyde. Finally, superfluous amino groups are blocked with, e.g., bovine serum albumin (BSA) so that antibody-fixed layer 14 is formed.

Method for Measuring an Amount of Protein Using an Antibody Chip

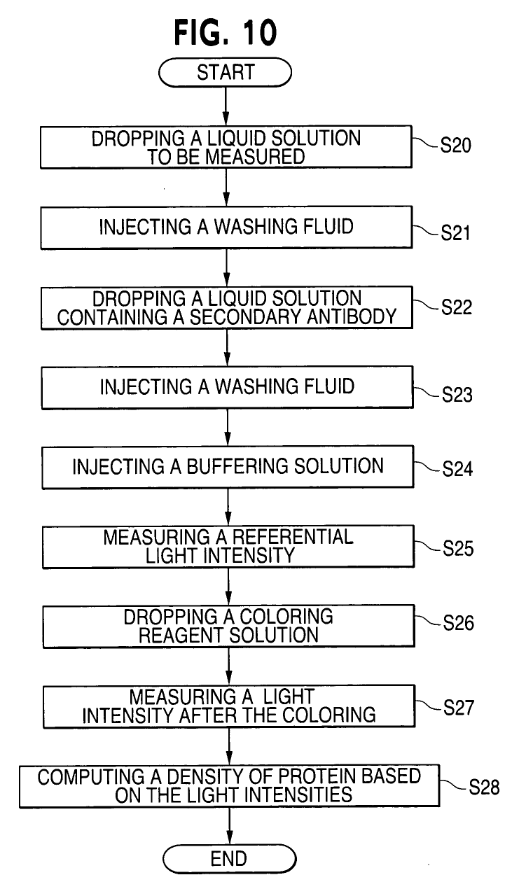

FIG. 10 is a flowchart showing steps used to measure an amount of protein with antibody chip 1 according to aspects of the present invention. In a first step (S20), a liquid solution to be measured is dropped on antibody-fixed layer 14 in reaction hole 10. In subsequent steps, a washing fluid is injected into cell 11 so that it flows into reaction hole 10 (S21), liquid solution containing an enzyme-labeled secondary antibody is dropped in reaction hole 10 (S22), a washing fluid is injected into cell 11 (S23), and a buffering solution is injected into cell 11 so that it flows into reaction hole 10 (S24). Next, a first light intensity is measured as a reference (S25) and a coloring reagent solution is dropped on the antibody-fixed layer 14 in reaction hole 10 (S26). Then, a second light intensity is measured (S27). Finally, a protein density is computed based on the variation of the first and second light intensities (S28). As one of ordinary skill in the art would appreciate, other steps may occur before, during, or after the steps shown in FIG. 10 or some of the steps shown in FIG. 10, e.g., the washing and buffering steps, may be re-ordered or deleted without departing from aspects of the present invention.

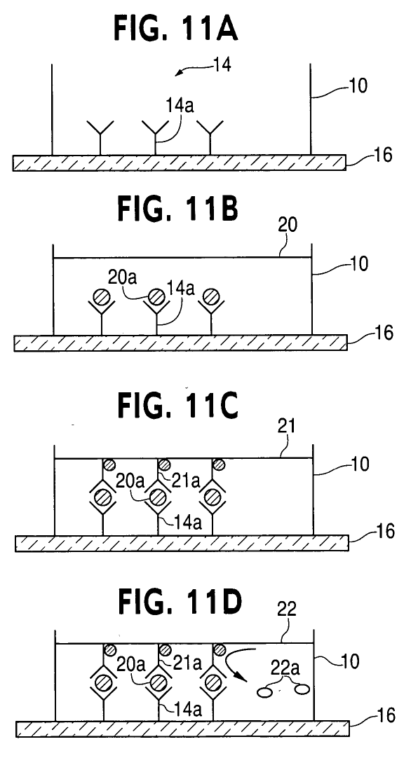

FIGS. 11A-11D illustrate certain steps according to the flowchart of FIG. 10.

(S20)

FIG. 11A shows an antibody-fixed layer 14 formed on the surface of substrate 16 in reaction hole 10 of antibody chip 1, as shown for example in FIG. 6. As shown in FIG. 11A, antibody-fixed layer 14 is made from primary antibodies 14a which specifically recognize antigens 20a whose amount is to be measured. Antigen 20a may be protein, peptide, nucleic acid, and so on. As shown in FIG. 11B, when a solution 20 comprising antigens 20a is dropped on antibody-fixed layer 14 in reaction hole 10, antigen 20a is bound to primary antibody 14a so that a complex between antigen 20a and primary antibody 14a is formed.

(S21)

Solution 20 may be washed with a washing fluid, e.g., phosphate buffered saline (PBS) including a surface-active agent.

(S22)

Then, the solution containing enzyme-labeled secondary antibody 21 is dropped on antibody-fixed layer 14 in reaction hole 10. As shown in FIG. 11C, enzyme-labeled secondary antibody 21a binds to a different portion of antigen 20a than antibody 14a. Finally, a complex of primary antibody 14a, antigen 20a, and enzyme-labeled secondary antibody 21a is formed. As a labeling enzyme, e.g., an oxidation-reduction enzyme, peroxidase (POD) may be used.

(S23)

Enzyme-labeled secondary antibody solution 21 comprising a superfluous enzyme-labeled secondary antibody 21a which is not bound to antigen 20a, is washed using a washing fluid such as PBS with a surface-active agent.

(S24)

Buffering solution, e.g., PBS, is injected into cell 11 so that it flows into reaction hole 10 to remove the used washing fluid with surface-active agent and to improve stability.

(S25)

Light emitting element 109 emits a light beam such as a laser beam toward incident grating 13a. The emitted light beam propagates inside substrate 16, and is output from outputting grating 13b. Light receiving element 110 receives the output light beam so that a referential intensity of the light is measured.

(S26)

As shown in FIG. 11D, a coloring reagent solution 22 is dropped in reaction hole 10. As an example of a coloring reagent solution 22, 80 m mol of acetic acid, 1.13 m mol of tetramethyl benzidine, 1.91 m mol of hydrogen peroxide (H₂O₂), and dimethyl sulfoxided (DMSO) (less than 1 wt. %) are preferably contained in 1 liter of buffering solution having pH 4.9.

An oxidation reduction reaction occurs between a labeling enzyme such as POD from enzyme-labeled secondary antibody 21a and H₂O₂ from coloring reagent solution 22 where H₂O₂ is a substrate of the labeling enzyme. As a result of that reaction, a radical oxygen atom (O*) is generated. Such a radical oxygen atom O* colors a coloring reagent 22a (an enzyme reaction product) contained in coloring reagent solution 22.

(S27)

After the coloring reagent 22a is produced, a light beam such as a laser beam is emitted from light emitting element 109 toward incident grating 13a in a similar fashion to step S25. The emitted light beam propagates inside substrate 16 and is output from outputting grating 13b. Then, the light beam from outputting grating 13b is detected by light receiving element 110 so that an intensity of the light after the coloring is measured.

(S28)

The density of antigen 20a in solution 20 is computed based on the difference between the reference intensity of light acquired in step S25 and the intensity of light after the coloring acquired in stepS27.

The density of antigen 20a is computed based on the referential intensity at a predetermined time A, and the intensity of the light after the coloring at a predetermined time B. It is not necessary to perform a control experiment using a liquid solution with no antigen in order to obtain a referential intensity.

Antibody chip 1 has reaction hole 10 surrounded by cell wall 12 where the antigen-antibody reaction and coloring reaction occur so that 1 micro liter of solution 20 is sufficient for the reactions.

Numerous modifications of the present invention are possible in light of the above teachings. It is therefore to be understood that, within the scope of the appended claims, the present invention can be practiced in a manner other than as specifically described herein.

1. An antigen measuring device, comprising:
an antibody chip which includes
a substrate,
a first grating on the surface of the substrate,
a second grating on the surface of the substrate,
an antibody-fixed layer on the surface of the substrate between the first and the second gratings,
a wall on the surface of the substrate, wherein the wall surrounds the antibody-fixed layer;
a light emitting element to emit a light beam toward the first grating; and
a light receiving element to receive the light beam which propagates through the substrate under the antibody-fixed layer and is output from the second grating.

2. An antigen measuring device according to claim 1, further comprising an injection tube connected to an injection pump to inject a liquid solution into an area surrounded by the wall.

3. An antigen measuring device according to claim 2, further comprising an evacuation tube connected to an evacuation pump to evacuate the liquid solution from the area surrounded by the wall.

4. An antigen measuring device according to claim 3, further comprising a lid which covers the antibody chip and fixes the position of the injection and evacuation tubes.

5. An antigen measuring device according to claim 4, further comprising one or more containers to contain solutions, and a valve connected to the injection pump, which valve selects a solution to be injected into the area surrounded by the wall.

6. An antigen measuring device according to claim 5, wherein the injection pump is configured to operate only when the antibody chip is installed in the antigen measuring device.

7. An antigen measuring device according to claim 6, wherein the light emitting element is configured to operate only when the antibody chip is closed by the lid.

8. An antigen measuring device according to claim 7, further comprising a light blocking plate projecting from the inner surface of the lid toward the antibody chip.

9. An antigen measuring device according to claim 8, further comprising a first pallet to set one or more antibody chips.

10. An antigen measuring device according to claim 8, further comprising plural pallets to respectively set a plurality of antibody chips, wherein the pallets are connected to each other.

11. A method of measuring antigen, comprising:
dropping a first liquid solution containing an antigen on an antibody-fixed layer formed on a substrate of an antibody chip to form a complex of the antibody and the antigen, which antibody chip includes a first and second gratings on the substrate;
dropping a second liquid solution containing a secondary antibody labeled by a labeling enzyme to form a complex of the antibody, the antigen, and the secondary antibody;
firstly measuring the intensity of a first light beam which propagates toward the first grating, through the substrate, and from the second grating;
dropping a coloring reagent solution on the antibody-fixed layer to produce an enzyme reaction product colored by a reaction with the labeling enzyme;
secondarily measuring the intensity of a second light beam which propagates toward the first grating, through the substrate, and from the second grating; and
measuring the antigen based on the variation between the intensities of the first and second light beams.

12. A method of measuring antigen according to claim 11, wherein measuring the antigen includes computing the density of the antigen based on the difference of the intensities of the first and second light beams.

13. A method of measuring antigen according to claim 11, wherein firstly measuring the intensity of a first light beam includes measuring the first light beam while injecting a buffering solution on the antibody-fixed layer.

14. A method of measuring antigen according to claim 11, wherein dropping the second liquid solution includes dropping the second liquid solution containing peroxidase.

15. A method of measuring antigen according to claim 11, wherein dropping the coloring reagent solution includes dropping the coloring reagent solution containing tetramethyl benzidine.

16. A method of measuring antigen according to claim 1 1, wherein dropping the coloring reagent solution includes dropping the coloring reagent solution containing tetramethyl benzidine and hydrogen peroxide.

17. A pallet with plural indentations to respectively set a plurality of antibody chips, wherein the distance between the center of the indentations is 9 mm.

18. An antibody chip package, comprising:
a chip container to set plural antibody chips at a constant interval;
a desiccant to keep the antibody chips dry; and
a package cover bound to the chip container.

19. An antibody chip package according to claim 18, wherein the distance between the center of the antibody chips is 9 mm when the antibody chips are set in the antibody chip package.
